(19) United States
(12) Patent Application Publication
(10) Pub. No.: US 2025/0235106 A1
(43) Pub. Date: Jul. 24, 2025

(54) INFORMATION PROCESSING APPARATUS, INFORMATION PROCESSING METHOD, AND PROGRAM

(71) Applicant: FUJIFILM Corporation, Tokyo (JP)

(72) Inventors: Atsushi INOUE, Tokyo (JP); Osamu MIYAZAKI, Tokyo (JP); Xinran ZHANG, Tokyo (JP); Takashi SHIRAHATA, Tokyo (JP); Koji YAMAGUCHI, Tokyo (JP)

(73) Assignee: FUJIFILM Corporation, Tokyo (JP)

(21) Appl. No.: 19/019,316

(22) Filed: Jan. 13, 2025

(30) Foreign Application Priority Data
Jan. 24, 2024 (JP) 2024-009041

(51) Int. Cl. A61B 5/00; A61B 5/107; G06T 7/70; G16H 30/20

(52) U.S. Cl. CPC A61B 5/0037; A61B 5/1072; A61B 5/4872; G06T 7/70; G16H 30/20

(57) ABSTRACT

There are provided an information processing apparatus, an information processing method, and a program which can acquire a tomographic image at an appropriate imaging position according to a body type of a subject.

The information processing apparatus includes at least one processor. The processor derives a determination target position of a subject in a body axis direction on the basis of body information indicating a physical feature of the subject in a predetermined range along the body axis direction of the subject, derives a reference position of the subject in the body axis direction on the basis of a subject image that is an image captured for the subject, and determines an imaging position of a tomographic image on the basis of a relative positional relationship between the determination target position and the reference position.

### CROSS-REFERENCE TO RELATED APPLICATIONS

The present application claims priority under 35 U.S.C. § 119 to Japanese Patent Application No. 2024-009041 filed on Jan. 24, 2024. The above application is hereby expressly incorporated by reference, in its entirety, into the present application.

### BACKGROUND

### 1. Technical Field

The disclosed technology relates to an information processing apparatus, an information processing method, and a program.

### 2. Description of the Related Art

The following technology is known for measuring visceral fat of a subject. JP2004-254932A discloses a technology of creating a three-dimensional image of a body surface of a subject on the basis of an X-ray CT image, designating a position corresponding to a predetermined visceral fat percentage measurement position on the body surface of the subject, on the three-dimensional image of the body surface, acquiring the X-ray CT image at the designated visceral fat percentage measurement position, and using the acquired X-ray CT image to obtain a visceral fat percentage of the subject.

JP2004-254933A discloses a technology of capturing a two-dimensional X-ray transmission image for imaging plan preparation for a subject to which a marker made of an X-ray shielding material is attached at a predetermined visceral fat percentage measurement position on a body surface of the subject, designating an appearance position of the marker on the two-dimensional X-ray transmission image as an imaging position for obtaining an X-ray CT image, and using the X-ray CT image of the designated appearance position of the marker to obtain a visceral fat percentage of the subject.

### SUMMARY

The metabolic syndrome is a syndrome in which at least two or more metabolic abnormalities are observed among three of high blood pressure, high blood sugar, and dyslipidemia, along with the accumulation of visceral fat, and early diagnosis and improvement of lifestyle are recommended. As one of diagnosing methods of the metabolic syndrome, a method of measuring visceral fat area using a tomographic image at a position of an umbilicus of the subject imaged by a computed tomography (CT) apparatus is adopted.

However, a person with an obese body type has a lower position of the umbilicus than a person with a standard body type, and the pelvis enters the tomographic image at the position of the umbilicus. Therefore, it is not appropriate to measure the visceral fat area of a person with an obese body type using the tomographic image at the position of the umbilicus. For the subject in which the fat accumulation is remarkable and the position of the umbilicus is biased downward, for example, it is preferable to measure the visceral fat area on the basis of the tomographic image at a height of a midpoint between the lower costal margin and the anterior superior iliac spine.

The disclosed technology has been made in view of the above-described points, and an object of the disclosed technology is to acquire a tomographic image at an appropriate imaging position according to a body type of a subject.

An information processing apparatus according to an aspect of the disclosed technology includes at least one processor. The processor derives a determination target position of a subject in a body axis direction on the basis of body information indicating a physical feature of the subject in a predetermined range along the body axis direction of the subject, derives a reference position of the subject in the body axis direction on the basis of a subject image that is an image captured for the subject, and determines an imaging position of a tomographic image on the basis of a relative positional relationship between the determination target position and the reference position.

The body information may include a body thickness distribution along the body axis direction of the subject. In this case, the processor may derive a position at which a body thickness of the subject in the body axis direction specified from the body thickness distribution is a largest, as the determination target position. The processor may acquire a distance image captured for the subject, and derive the body thickness distribution on the basis of the distance image.

The processor may acquire an optical image captured for the subject, as the body information, and derive a position of an umbilicus of the subject specified from the optical image, as the determination target position.

The subject image may be a scano image. In this case, the processor may derive an uppermost portion of a pelvic region specified from the scano image in the body axis direction, as the reference position. The subject image may be an optical image. In this case, the processor may derive a position of a waist of the subject specified from the optical image, as the reference position.

The processor may determine a first position of the subject in the body axis direction as the imaging position in a case where the determination target position is on a lower side in the body axis direction with respect to the reference position, and determine a second position different from the first position as the imaging position in a case where the determination target position is on an upper side in the body axis direction with respect to the reference position.

In a case where the determination target position is on the lower side in the body axis direction with respect to the reference position, the processor may specify a position of an anterior superior iliac spine and a position of a lower costal margin of the subject on the basis of the subject image, and determine a center between the position of the anterior superior iliac spine and the position of the lower costal margin in the body axis direction, as the imaging position.

In a case where the determination target position is on the lower side in the body axis direction with respect to the reference position, the processor may specify a fourth lumbar vertebra of the subject on the basis of the subject image, and determine a center of the fourth lumbar vertebra in the body axis direction, as the imaging position.

In a case where the determination target position is on the lower side in the body axis direction with respect to the reference position, the processor may specify a position of a waist of the subject on the basis of the subject image, and determine a predetermined position in the body axis direction specified from the position of the waist, as the imaging position.

In a case where the determination target position is on the lower side in the body axis direction with respect to the reference position, the processor may specify a position of a shoulder and a position of a waist of the subject on the basis of the subject image, and determine a predetermined position in the body axis direction specified from both the position of the shoulder and the position of the waist, as the imaging position.

In a case where the determination target position is on an upper side in the body axis direction with respect to the reference position, the processor may determine the determination target position as the imaging position.

An information processing method according to another aspect of the disclosed technology is a method that is executed by at least one processor of an information processing apparatus, the method including: deriving a determination target position of a subject in a body axis direction on the basis of body information indicating a physical feature of the subject in a predetermined range along the body axis direction of the subject; deriving a reference position of the subject in the body axis direction on the basis of a subject image that is an image captured for the subject; and determining an imaging position of a tomographic image on the basis of a relative positional relationship between the determination target position and the reference position.

A program according to another aspect of the disclosed technology is a program for causing at least one processor of an information processing apparatus to execute processing including deriving a determination target position of a subject in a body axis direction on the basis of body information indicating a physical feature of the subject in a predetermined range along the body axis direction of the subject; deriving a reference position of the subject in the body axis direction on the basis of a subject image that is an image captured for the subject; and determining an imaging position of a tomographic image on the basis of a relative positional relationship between the determination target position and the reference position.

According to the disclosed technology, it is possible to acquire a tomographic image at an appropriate imaging position according to a body type of a subject.

### BRIEF DESCRIPTION OF THE DRAWINGS

Exemplary embodiments according to the technique of the present disclosure will be described in detail based on the following figures, wherein:

FIG. 1 is a diagram illustrating an example of a configuration of an imaging system 1 according to an embodiment of the disclosed technology;

FIG. 2 is a perspective view illustrating an example of a configuration of a CT apparatus according to an embodiment of the disclosed technology;

FIG. 3 is a cross-sectional view of a CT apparatus according to an embodiment of the disclosed technology;

FIG. 4 is a diagram illustrating an example of a hardware configuration of an information processing apparatus according to an embodiment of the disclosed technology;

FIG. 5 is a functional block diagram illustrating an example of a functional configuration of an information processing apparatus according to an embodiment of the disclosed technology;

FIG. 6 is a diagram illustrating an example of a body thickness distribution derived for a subject according to an embodiment of the disclosed technology;

FIG. 7 is a diagram illustrating an example of a scano image according to an embodiment of the disclosed technology;

FIG. 8 is a diagram illustrating a body thickness distribution and a scano image according to an embodiment of the disclosed technology;

FIG. 9 is a diagram illustrating an example of an aspect of deriving an imaging position of a tomographic image according to an embodiment of the disclosed technology;

FIG. 10 is a flowchart illustrating an example of a flow of processing performed by executing a processing program according to an embodiment of the disclosed technology;

FIG. 11 is a diagram illustrating an example of a configuration of an imaging system according to another embodiment of the disclosed technology;

FIG. 12 is a diagram illustrating an example of an optical image according to another embodiment of the disclosed technology;

FIG. 13 is a diagram illustrating a body thickness distribution and a scano image according to another embodiment of the disclosed technology; and

FIG. 14 is a diagram illustrating an example of an aspect of deriving an imaging position of a tomographic image according to another embodiment of the disclosed technology.

### DETAILED DESCRIPTION

Hereinafter, an example of embodiments of the disclosed technology will be described with reference to the drawings. Note that the same or equivalent constituent elements and portions in the drawings are assigned by the same reference numerals, and overlapping descriptions will be omitted.

### First Embodiment

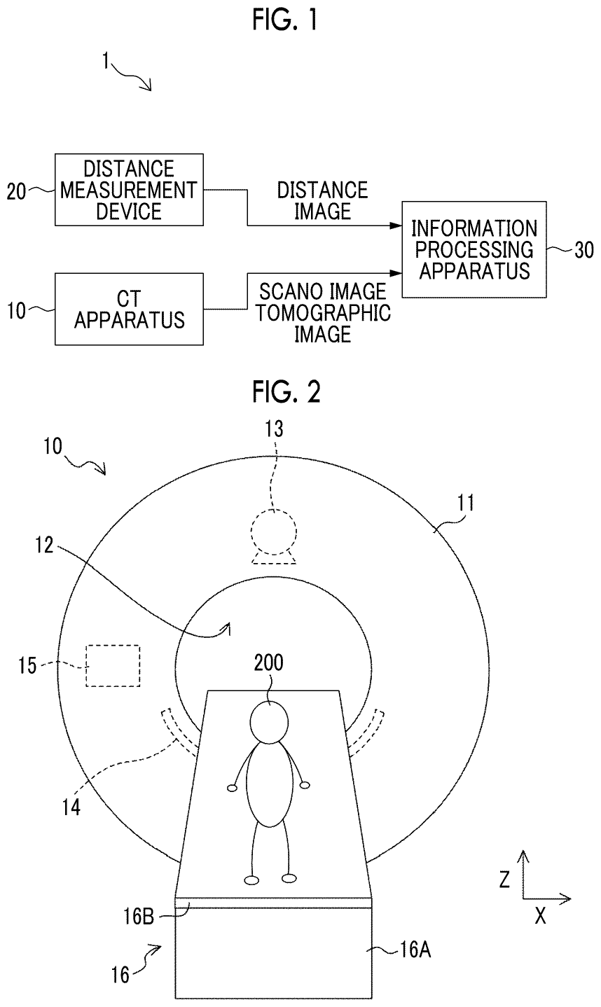

FIG. 1 is a diagram illustrating an example of a configuration of an imaging system 1 according to an embodiment of the disclosed technology. The imaging system 1 includes a CT apparatus 10, a distance measurement device 20, and an information processing apparatus 30. In the following, a case where the imaging system 1 is used for measuring visceral fat of a subject will be described as an example.

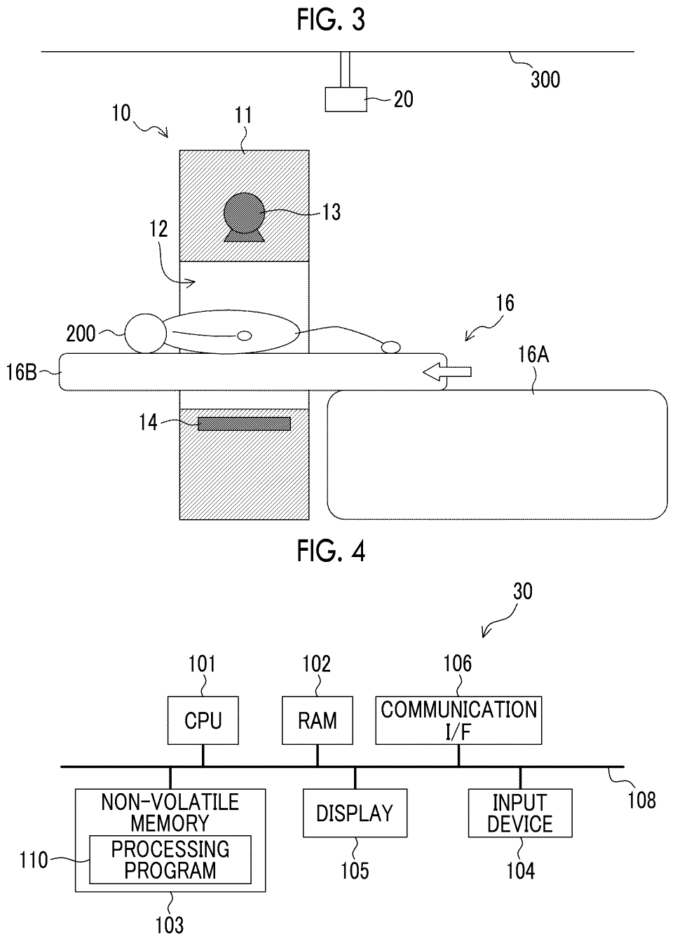

FIG. 2 is a perspective view illustrating an example of a configuration of the CT apparatus 10, and FIG. 3 is a cross-sectional view of the CT apparatus 10. The CT apparatus 10 includes a gantry 11 and an examination table 16. The gantry 11 has a tunnel-like structure having an opening portion 12 at the center thereof. A radiation source unit 13 that emits X-rays and a detection unit 14 that detects X-rays and generates a radiation image are provided inside the gantry 11. The radiation source unit 13 and the detection unit 14 can be each rotated along an annular shape of the gantry 11 while maintaining a positional relationship of facing each other. In addition, a controller 15 that controls an operation of the CT apparatus 10 is provided inside the gantry 11.

The examination table 16 includes a base portion 16A that is fixed to the floor, and a bed portion 16B on which a subject 200 lies. In a case where capturing of a radiation image is performed for the subject 200, the bed portion 16B is slid, and the subject 200 lying on the bed portion 16B is transported into an opening portion 12 of the gantry 11.

The CT apparatus 10 can capture a scano image and a tomographic image. The scano image is a radiation image including a predetermined range along a body axis direction of the subject 200. The scano image is captured by emitting the radiation from the radiation source unit 13 while maintaining a state in which the radiation source unit 13 is positioned directly above the subject 200 and sliding the bed portion 16B. In the CT apparatus 10, the capturing of the scano image is performed before the capturing of the tomographic image. The scano image is an example of a “subject image” in the disclosed technology.

The tomographic image is a radiation image in a cross section intersecting the body axis direction of the subject 200. The tomographic image is obtained by reconstructing a plurality of radiation images captured by irradiating the subject 200 with radiation from a plurality of different directions. As the reconstruction method, for example, a back projection method or a sequential approximate image reconstruction method can be used. The scano image and the tomographic image obtained by the CT apparatus 10 are transmitted to the information processing apparatus 30.

The distance measurement device 20 is an imaging apparatus that can generate a distance image representing a distance to a surface of an object. The distance measurement device 20 may be, for example, a distance measurement camera that generates a distance image representing a distance to a surface of an object by a time-of-flight method using infrared light. The distance measurement device 20 is disposed at a position where the body thickness distribution of the subject along the body axis direction can be acquired. As illustrated in FIG. 3, the distance measurement device 20 may be attached to, for example, a ceiling 300 in an imaging room in which the CT apparatus 10 is installed. By capturing the distance image while sliding the bed portion 16B, the distance image in a predetermined range along the body axis direction of the subject 200 is obtained. An imaging range of the distance measurement device 20 is substantially the same as the imaging range of the scano image. The distance image obtained by the distance measurement device 20 is transmitted to the information processing apparatus 30.

FIG. 4 is a diagram illustrating an example of a hardware configuration of the information processing apparatus 30. The information processing apparatus 30 includes a central processing unit (CPU) 101, a random-access memory (RAM) 102, a non-volatile memory 103, an input device 104 including a keyboard and a mouse, a display 105, and a communication interface 106. These pieces of hardware are connected to a bus 108.

The display 105 may be a touch panel display. The communication interface 106 is an interface for the information processing apparatus 30 to perform communication with the CT apparatus 10 and the distance measurement device 20. The communication method may be either wired or wireless. For the wireless communication, it is possible to apply a method conforming to existing wireless communication standards such as, for example, Wi-Fi (registered trademark) and Bluetooth (registered trademark).

The non-volatile memory 103 is a non-volatile storage medium such as a hard disk or a flash memory. A processing program 110 is stored in the non-volatile memory 103. The RAM 102 is a work memory for the CPU 101 to execute processing. The CPU 101 loads the processing program 110 stored in the non-volatile memory 103 into the RAM 102, and executes processing according to the processing program 110. The CPU 101 is an example of a “processor” in the disclosed technology.

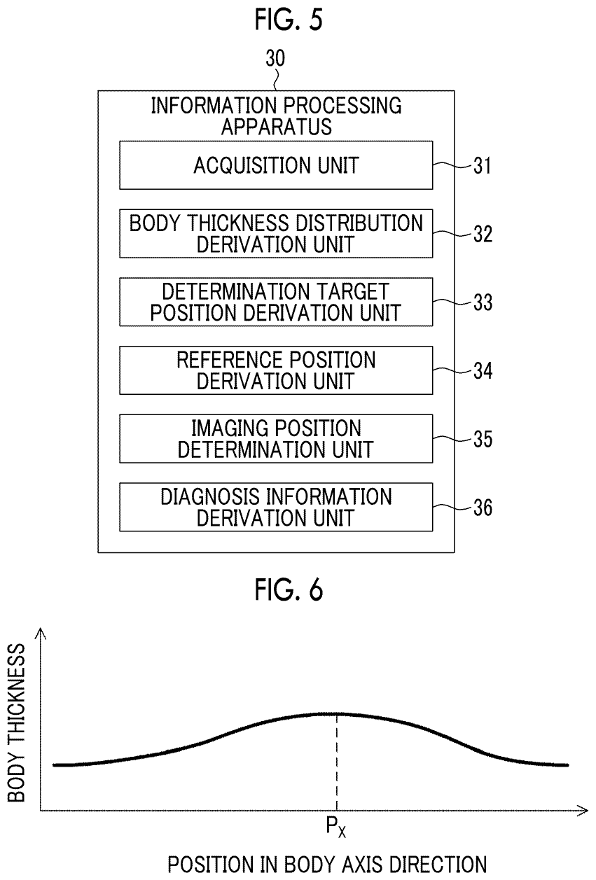

FIG. 5 is a functional block diagram illustrating an example of a functional configuration of the information processing apparatus 30. The information processing apparatus 30 includes an acquisition unit 31, a body thickness distribution derivation unit 32, a determination target position derivation unit 33, a reference position derivation unit 34, an imaging position determination unit 35, and a diagnosis information derivation unit 36. The CPU 101 executes the processing program 110, and thereby the information processing apparatus 30 functions as the acquisition unit 31, the body thickness distribution derivation unit 32, the determination target position derivation unit 33, the reference position derivation unit 34, the imaging position determination unit 35, and the diagnosis information derivation unit 36.

The acquisition unit 31 acquires the scano image captured by the CT apparatus 10. In addition, the acquisition unit 31 acquires the distance image captured by the distance measurement device 20.

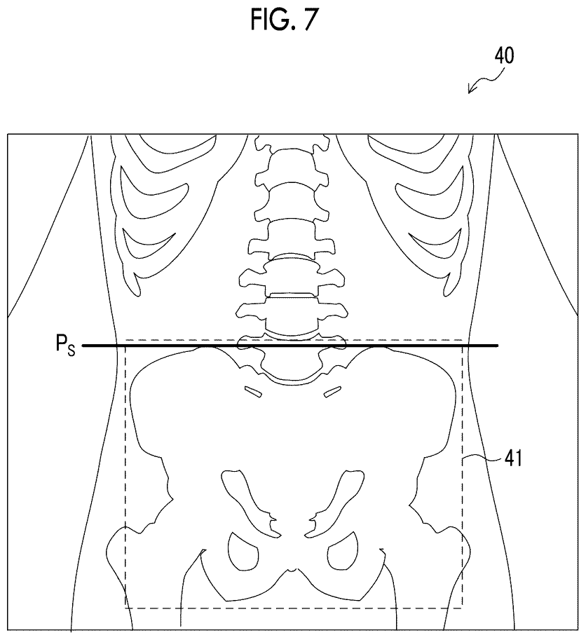

The body thickness distribution derivation unit 32 derives the body thickness distribution of the subject 200 along the body axis direction on the basis of the distance image acquired by the acquisition unit 31. FIG. 6 is a diagram illustrating an example of the body thickness distribution along the body axis direction derived for the subject 200. The body thickness distribution derivation unit 32 derives a difference (L1−L2) between a distance L1 from the distance measurement device 20 to the bed portion 16B and a distance L2 from the distance measurement device 20 to the body surface of the subject 200, which is indicated by the distance image, at each position of the subject 200 along the body axis direction, and outputs the difference as the body thickness distribution of the subject 200 along the body axis direction. Note that the body thickness distribution of the subject 200 along the body axis direction is an example of “body information” in the disclosed technology. The body information is information indicating a physical feature of the subject 200.

The determination target position derivation unit 33 derives a determination target position of the subject in the body axis direction on the basis of the body thickness distribution derived by the body thickness distribution derivation unit 32. The determination target position is a position of the subject 200 in the body axis direction for determining whether or not the subject 200 has an obese body type. As illustrated in FIG. 6, the determination target position derivation unit 33 derives a position at which the body thickness specified from the body thickness distribution of the subject 200 along the body axis direction is the largest as a determination target position P_{X}. It is estimated that the position at which the body thickness in the body axis direction is the largest is the position of the umbilicus of the subject 200.

The reference position derivation unit 34 derives the reference position of the subject 200 in the body axis direction on the basis of the scano image acquired by the acquisition unit 31. The reference position is a position of the subject 200 in the body axis direction, which is a reference for determining whether or not the subject 200 has an obese body type. In the present embodiment, the uppermost portion of a pelvic region in the body axis direction is set as the reference position. FIG. 7 is a diagram illustrating an example of a scano image 40 acquired by the acquisition unit 31. The reference position derivation unit 34 specifies a pelvic region 41 from the scano image 40. The reference position derivation unit 34 derives the uppermost portion of the pelvic region 41 specified from the scano image 40 in the body axis direction as a reference position P_{S}. Note that, as a technology of specifying the pelvic region 41 from the scano image 40, for example, an image recognition technology using machine learning can be used. In addition, the pelvic region 41 may be specified by detecting the feature point from the profile.

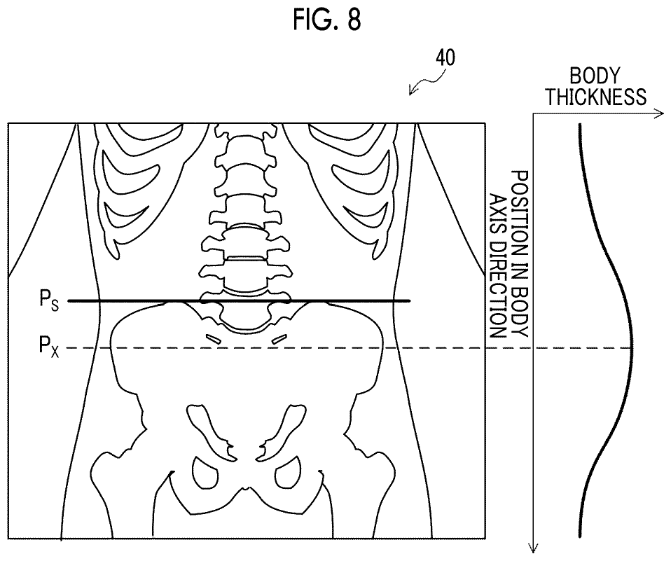

The imaging position determination unit 35 determines the imaging position of the tomographic image captured by the CT apparatus 10 on the basis of a relative positional relationship between the determination target position derived by the determination target position derivation unit 33 and the reference position derived by the reference position derivation unit 34. As illustrated in FIG. 8, the imaging position determination unit 35 performs positioning of the body thickness distribution of the subject 200 along the body axis direction and the scano image 40 in the body axis direction.

In a case where the determination target position P_{X} (position of the umbilicus) is on the lower side (foot side) in the body axis direction with respect to the reference position P_{S} (uppermost portion of the pelvic region), the imaging position determination unit 35 determines a first position in the body axis direction of the subject as the imaging position of the tomographic image. In a case where the determination target position P_{X} (position of the umbilicus) is on the lower side (foot side) in the body axis direction with respect to the reference position P_{S} (uppermost portion of the pelvic region), it is estimated that the position of the umbilicus of the subject 200 is lower than that of a person with a standard body type and the body type of the subject 200 is an obese body type. In this case, the first position determined as the imaging position of the person with the obese body type is determined as the imaging position of the tomographic image.

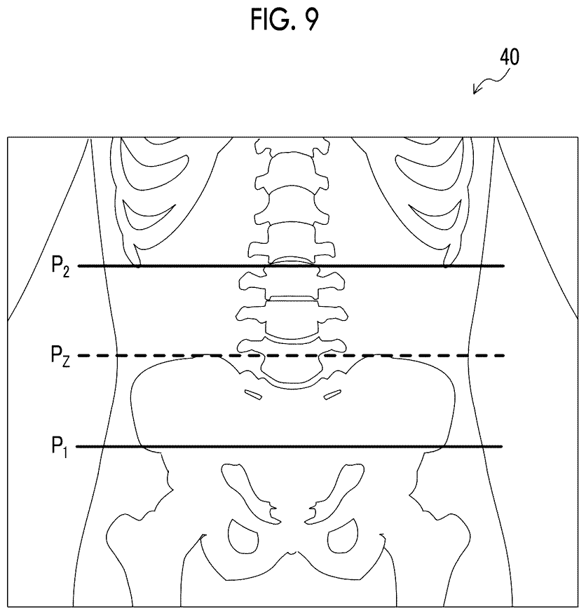

More specifically, in a case where the determination target position P_{X} is on the lower side (foot side) in the body axis direction with respect to the reference position P_{S}, the imaging position determination unit 35 specifies a position P₁ of the anterior superior iliac spine and a position P₂ of the lower costal margin of the subject 200 on the basis of the scano image 40 as illustrated in FIG. 9. The imaging position determination unit 35 determines a center in the body axis direction between the position P₁ of the anterior superior iliac spine and the position P₂ of the lower costal margin, as an imaging position P_{Z} of the tomographic image. Note that, as a technology of specifying the positions P₁ and P₂, for example, an image recognition technology using machine learning can be used. In addition, the positions P₁ and P₂ may be specified by detecting the feature point from the profile.

On the other hand, in a case where the determination target position P_{X} (position of the umbilicus) is on the upper side (head side) in the body axis direction with respect to the reference position P_{S} (uppermost portion of the pelvic region), the imaging position determination unit 35 determines a second position in the body axis direction of the subject as the imaging position of the tomographic image. In a case where the determination target position P_{X} (position of the umbilicus) is on the upper side (head side) in the body axis direction with respect to the reference position P_{S} (uppermost portion of the pelvic region), it is estimated that the body type of the subject 200 is a standard body type. In this case, the second position that is different from the first position and is determined as the imaging position of a person with the standard body type is determined as the imaging position of the tomographic image.

More specifically, in a case where the determination target position P_{X} is on the upper side (head side) in the body axis direction with respect to the reference position P_{S}, the imaging position determination unit 35 determines the position of the umbilicus of the subject 200 as the imaging position of the tomographic image. In the present embodiment, since the determination target position P_{X} corresponds to the position of the umbilicus of the subject 200, the imaging position determination unit 35 determines the determination target position P_{X} as the imaging position of the tomographic image in a case where the determination target position P_{X} is on the upper side (head side) in the body axis direction with respect to the reference position P_{S}.

The imaging position determination unit 35 transmits an imaging instruction for acquiring a tomographic image at the determined imaging position, to the CT apparatus 10. The CT apparatus 10 captures a radiation image of the subject 200 on the basis of the imaging instruction, and generates a tomographic image at the determined imaging position. The acquisition unit 31 acquires the tomographic image generated by the CT apparatus 10.

The diagnosis information derivation unit 36 derives diagnosis information on the basis of the tomographic image generated in the CT apparatus 10. Specifically, the diagnosis information derivation unit 36 specifies a visceral fat portion from the tomographic image, and derives information indicating the area of the specified visceral fat portion as the diagnosis information. As a technology of specifying the visceral fat portion from the tomographic image, for example, an image recognition technology using machine learning can be used.

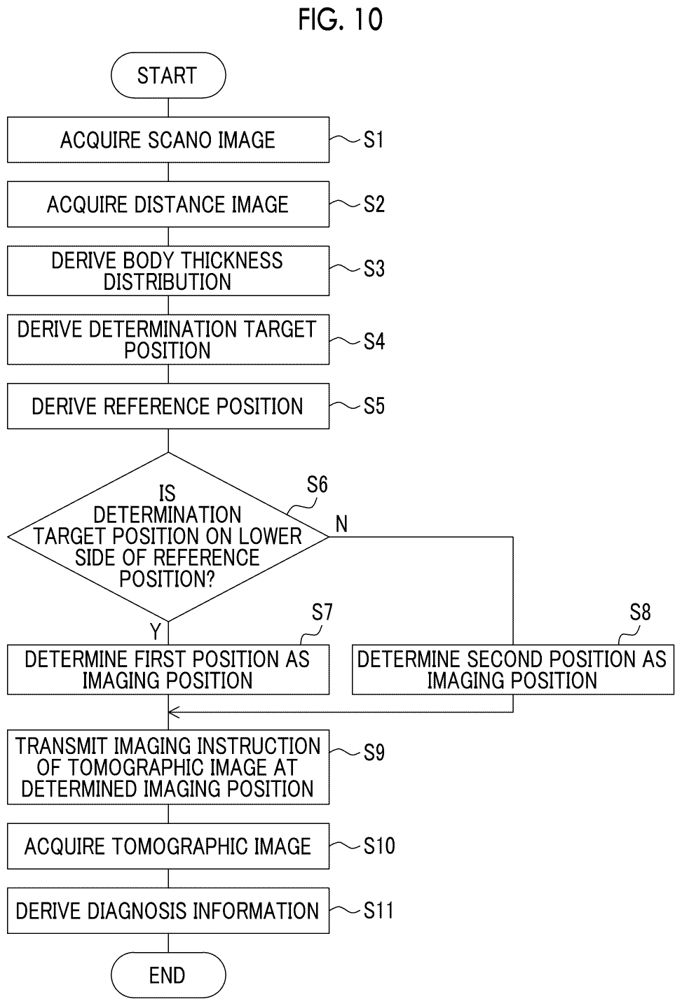

FIG. 10 is a flowchart illustrating an example of a flow of processing performed by the CPU 101 executing the processing program 110.

In step S1, the acquisition unit 31 acquires the scano image captured by the CT apparatus 10. In step S2, the acquisition unit 31 acquires the distance image captured by the distance measurement device 20.

In step S3, the body thickness distribution derivation unit 32 derives a body thickness distribution of the subject 200 along the body axis direction on the basis of the distance image acquired in step S2. The body thickness distribution derivation unit 32 derives the difference (L1−L2) between the distance L1 from the distance measurement device 20 to the bed portion 16B and the distance L2 from the distance measurement device 20 to the body surface of the subject 200, which is indicated by the distance image, at each position of the subject 200 along the body axis direction, and outputs the difference as the body thickness distribution of the subject 200 along the body axis direction.

In step S4, the determination target position derivation unit 33 derives the determination target position of the subject in the body axis direction on the basis of the body thickness distribution derived in step S3. The determination target position derivation unit 33 derives the position at which the body thickness specified from the body thickness distribution of the subject 200 along the body axis direction is the largest, as the determination target position P_{X} (refer to FIG. 6). It is estimated that the position at which the body thickness in the body axis direction is the largest is the position of the umbilicus of the subject 200.

In step S5, the reference position derivation unit 34 derives the reference position of the subject 200 in the body axis direction on the basis of the scano image acquired in step S1. The reference position derivation unit 34 derives the uppermost portion of the pelvic region 41 in the body axis direction as the reference position P_{S} (refer to FIG. 7).

In step S6, the imaging position determination unit 35 determines whether or not the determination target position derived in step S4 is on the lower side (foot side) in the body axis direction with respect to the reference position derived in step S5. In a case where it is determined that the determination target position is on the lower side (foot side) in the body axis direction with respect to the reference position, the processing proceeds to step S7, and in a case where it is determined that the determination target position is on the upper side (head side) in the body axis direction with respect to the reference position, the processing proceeds to step S8.

In step S7, the imaging position determination unit 35 determines the first position, which is determined as the imaging position of the person with the obese body type, as the imaging position of the tomographic image. Specifically, the imaging position determination unit 35 determines the center in the body axis direction between the position P₁ of the anterior superior iliac spine and the position P₂ of the lower costal margin, as the imaging position P_{Z} of the tomographic image (refer to FIG. 9).

In step S8, the imaging position determination unit 35 determines the second position, which is determined as the imaging position of the person with the standard body type, as the imaging position of the tomographic image. Specifically, the imaging position determination unit 35 determines the position of the umbilicus of the subject, that is, the determination target position derived in step S4, as the imaging position of the tomographic image.

In step S9, the imaging position determination unit 35 transmits an imaging instruction for acquiring a tomographic image at the imaging position determined in step S7 or step S8, to the CT apparatus 10. The CT apparatus 10 captures a radiation image of the subject 200 on the basis of the imaging instruction, and generates a tomographic image at the imaging position determined in step S7 or step S8. In step S10, the acquisition unit 31 acquires the tomographic image generated by the CT apparatus 10.

In step S11, the diagnosis information derivation unit 36 derives diagnosis information on the basis of the tomographic image acquired in step S10. Specifically, the diagnosis information derivation unit 36 specifies a visceral fat portion from the tomographic image, and derives information indicating the area of the specified visceral fat portion as the diagnosis information.

As described above, the information processing apparatus 30 according to the embodiment of the disclosed technology derives the determination target position of the subject in the body axis direction on the basis of the body information indicating the physical feature of the subject in a predetermined range along the body axis direction of the subject, derives the reference position of the subject in the body axis direction on the basis of the subject image which is the image captured for the subject, and determines the imaging position of the tomographic image on the basis of the relative positional relationship between the determination target position and the reference position.

The relative positional relationship between the determination target position and the reference position is changed according to the body type of the subject. However, according to the information processing apparatus 30 according to the embodiment of the disclosed technology, since the imaging position of the tomographic image is determined on the basis of the relative positional relationship between the determination target position and the reference position, it is possible to acquire a tomographic image at an appropriate imaging position according to the body type of the subject.

Note that, in the above description, the case where the body thickness distribution is derived on the basis of the distance image captured by the distance measurement device 20 has been described as an example, but the disclosed technology is not limited to this aspect. The body thickness distribution may be measured on the basis of one-dimensional distance information along the body axis direction of the subject. In this case, the distance measurement device 20 may output one-dimensional distance measurement information indicating a distance to each point along the body axis direction of the subject.

In addition, in the above description, the case where the body thickness distribution along the body axis direction of the subject is used as the body information indicating the physical feature of the subject 200 has been described as an example, but the disclosed technology is not limited to this aspect. The information processing apparatus 30 may acquire the optical image captured for the subject, as the body information of the subject. The optical image may be acquired by a general digital camera. For example, in a case where the optical image captured for the subject in a state of undressing is acquired as the body information, the determination target position derivation unit 33 may derive a position of the umbilicus of the subject specified from the optical image as the determination target position. In addition, in a case where the optical image captured for the subject in a state of wearing the clothes is acquired as the body information, the determination target position derivation unit 33 may derive a position of the umbilicus of the subject estimated by machine learning or the like from the optical image as the determination target position.

In addition, in the above description, the case where the center between the position of the anterior superior iliac spine and the position of the lower costal margin in the body axis direction is determined as the imaging position of the tomographic image in a case where the determination target position is on the lower side with respect to the reference position has been described as an example, but the disclosed technology is not limited to this aspect. In a case in which the determination target position is on the lower side with respect to the reference position, the center of the fourth lumbar vertebra in the body axis direction may be determined as the imaging position. In this case, the imaging position determination unit 35 specifies the fourth lumbar vertebra of the subject on the basis of the scano image, and determines the center of the fourth lumbar vertebra in the body axis direction as the imaging position.

### Second Embodiment

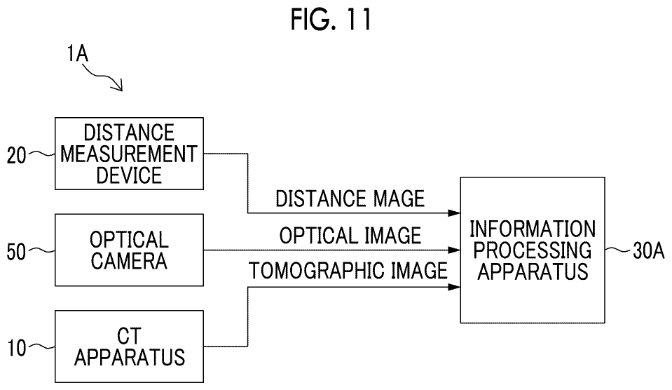

FIG. 11 is a diagram illustrating an example of a configuration of an imaging system 1A according to a second embodiment of the disclosed technology. The imaging system 1A includes the CT apparatus 10, the distance measurement device 20, an optical camera 50, and an information processing apparatus 30A.

The optical camera 50 is an imaging device that can capture an optical image. The optical camera may be a general digital camera. An optical image including a predetermined range along the body axis direction of the subject 200 is captured by the optical camera 50. The optical image captured by the optical camera 50 is an example of a “subject image” in the disclosed technology.

Similarly to the information processing apparatus 30 according to the first embodiment, the information processing apparatus 30A according to the present embodiment includes the acquisition unit 31, the body thickness distribution derivation unit 32, the determination target position derivation unit 33, the reference position derivation unit 34, the imaging position determination unit 35, and the diagnosis information derivation unit 36 (refer to FIG. 5).

The acquisition unit 31 acquires the distance image captured by the distance measurement device 20 and the optical image captured by the optical camera 50. The body thickness distribution derivation unit 32 derives the body thickness distribution of the subject 200 along the body axis direction on the basis of the distance image acquired by the acquisition unit 31, similarly to the information processing apparatus 30 according to the first embodiment. Similarly to the information processing apparatus 30 according to the first embodiment, the determination target position derivation unit 33 derives the position at which the body thickness specified from the body thickness distribution of the subject 200 along the body axis direction is the largest, as the determination target position P_{X} (refer to FIG. 6).

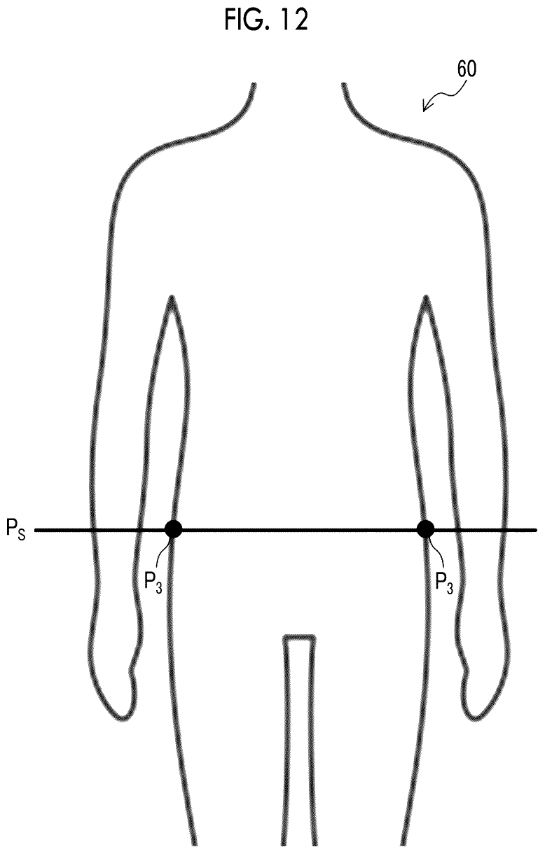

The reference position derivation unit 34 derives the reference position of the subject 200 in the body axis direction on the basis of the optical image acquired by the acquisition unit 31. The reference position in the present embodiment is the position of the waist. FIG. 12 is a diagram illustrating an example of an optical image 60 acquired by the acquisition unit 31. The reference position derivation unit 34 specifies a position P₃ of the waist from the optical image 60. As a technology of specifying the position P₃ of the waist from the optical image 60, for example, an image recognition technology using machine learning can be used. In addition, the position P₃ of the waist may be specified by detecting the feature point from the profile. The reference position derivation unit 34 derives the position of the waist specified from the optical image 60 in the body axis direction as the reference position P_{S}.

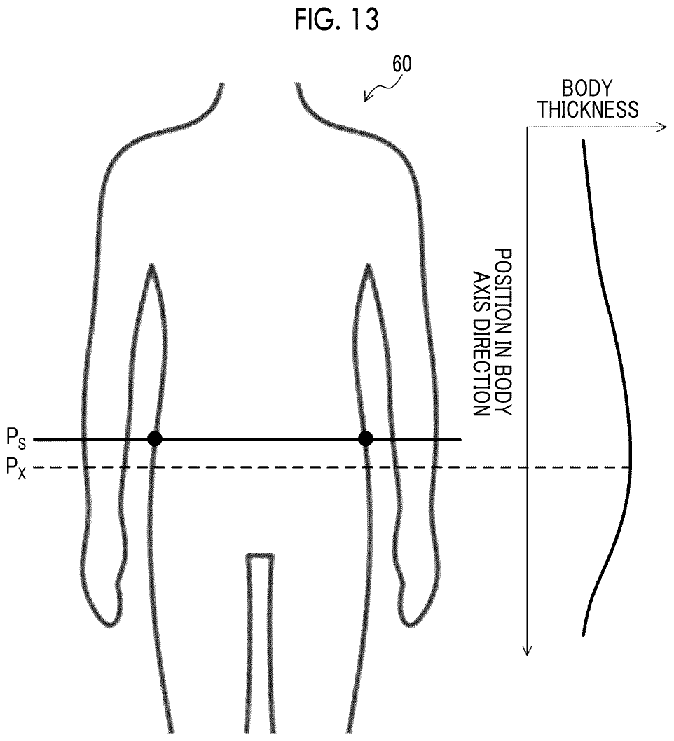

The imaging position determination unit 35 determines the imaging position of the tomographic image captured by the CT apparatus 10 on the basis of a relative positional relationship between the determination target position derived by the determination target position derivation unit 33 and the reference position derived by the reference position derivation unit 34. As illustrated in FIG. 13, the imaging position determination unit 35 performs positioning of the body thickness distribution of the subject 200 along the body axis direction and the optical image 60 in the body axis direction.

In a case where the determination target position P_{X} (position of the umbilicus) is on the lower side (foot side) in the body axis direction with respect to the reference position P_{S} (position of the waist), the imaging position determination unit 35 determines the first position of the subject in the body axis direction as the imaging position of the tomographic image. In a case where the determination target position P_{X} (position of the umbilicus) is on the lower side (foot side) in the body axis direction with respect to the reference position P_{S} (position of the waist), it is estimated that the position of the umbilicus of the subject 200 is lower than that of a person with a standard body type and the body type of the subject 200 is an obese body type. In this case, the first position determined as the imaging position of the person with the obese body type is determined as the imaging position of the tomographic image.

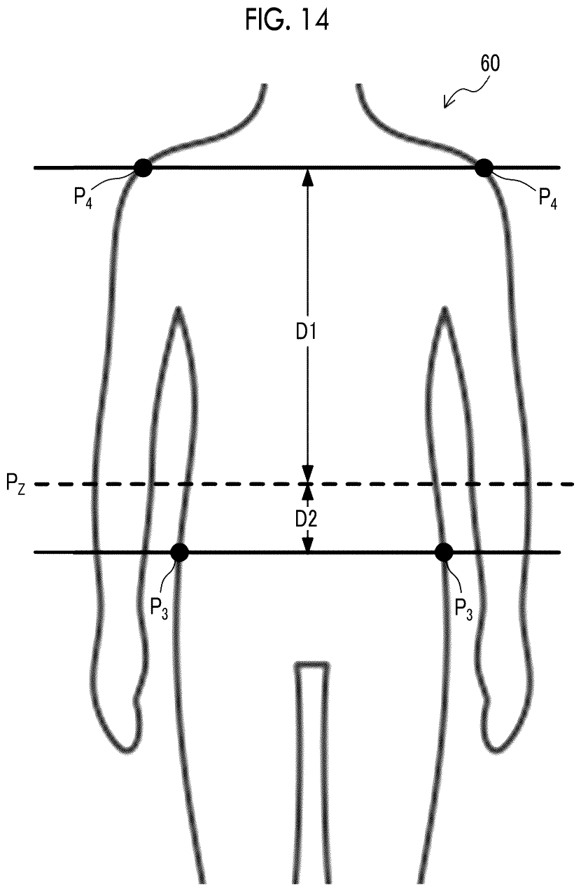

More specifically, in a case where the determination target position P_{X} is on the lower side (foot side) in the body axis direction with respect to the reference position P_{S}, the imaging position determination unit 35 specifies a position P₄ of the shoulder in addition to the position P₃ of the waist of the subject 200 on the basis of the optical image 60 as illustrated in FIG. 14. As a technology of specifying the position P₃ of the waist and the position P₄ of the shoulder, for example, an image recognition technology using machine learning can be used. In addition, the position P₃ of the waist and the position P₄ of the shoulder may be specified by detecting the feature point from the profile. The imaging position determination unit 35 determines a predetermined position in the body axis direction specified from both the position P₃ of the waist and the position P₄ of the shoulder, as the imaging position P_{Z} of the tomographic image. For example, a position in the body axis direction at which a ratio (D1:D2) of a distance D1 from the position P₃ of the waist and a distance D2 from the position P₄ of the shoulder is a predetermined ratio (for example, 1:9) may be determined as the imaging position P_{Z}.

On the other hand, in a case where the determination target position P_{X} (position of the umbilicus) is on the upper side (head side) in the body axis direction with respect to the reference position P_{S} (position of the waist), the imaging position determination unit 35 determines the second position of the subject in the body axis direction as the imaging position of the tomographic image. In a case where the determination target position P_{X} (position of the umbilicus) is on the upper side (head side) in the body axis direction with respect to the reference position P_{S} (position of the waist), it is estimated that the body type of the subject 200 is a standard body type. In this case, the second position that is different from the first position and is determined as the imaging position of a person with the standard body type is determined as the imaging position of the tomographic image.

More specifically, in a case where the determination target position P_{X} is on the upper side (head side) in the body axis direction with respect to the reference position P_{S}, the imaging position determination unit 35 determines the position of the umbilicus of the subject 200, as the imaging position of the tomographic image. In the present embodiment, since the determination target position P_{X} corresponds to the position of the umbilicus of the subject 200, the imaging position determination unit 35 determines the determination target position P_{X} as the imaging position of the tomographic image in a case where the determination target position P_{X} is on the upper side (head side) in the body axis direction with respect to the reference position P_{S}.

Similarly to the information processing apparatus 30 according to the first embodiment, the imaging position determination unit 35 transmits an imaging instruction for acquiring a tomographic image at the determined imaging position, to the CT apparatus 10. The CT apparatus 10 captures a radiation image of the subject 200 on the basis of the imaging instruction, and generates a tomographic image at the determined imaging position. The acquisition unit 31 acquires the tomographic image generated by the CT apparatus 10.

Similarly to the information processing apparatus 30 according to the first embodiment, the diagnosis information derivation unit 36 derives diagnosis information on the basis of the tomographic image generated in the CT apparatus 10. Specifically, the diagnosis information derivation unit 36 specifies a visceral fat portion from the tomographic image, and derives information indicating the area of the specified visceral fat portion as the diagnosis information.

As described above, with the information processing apparatus 30A according to the present embodiment, as in the information processing apparatus 30 according to the first embodiment, since the imaging position of the tomographic image is determined on the basis of the relative positional relationship between the determination target position and the reference position, it is possible to acquire a tomographic image at an appropriate imaging position according to the body type of the subject.

In addition, with the information processing apparatus 30A according to the present embodiment, the reference position and the imaging position are derived on the basis of the optical image. That is, it is not necessary to use the scano image for deriving the reference position and the imaging position. Therefore, it is possible to suppress the amount of radiation exposure of the subject as compared with the information processing apparatus 30 according to the first embodiment.

Note that, in the above description, the case where the predetermined position in the body axis direction at which the ratio of the distance from the position P₃ of the waist and the distance from the position P₄ of the shoulder is the predetermined ratio is determined as the imaging position of the tomographic image has been described as an example, but the disclosed technology is not limited to this aspect. The imaging position determination unit 35 may specify a central position in the body axis direction between the position of the anterior superior iliac spine and the position of the lower costal margin from the position P₃ of the waist and the position P₄ of the shoulder, and may determine the position as the imaging position P_{Z} of the tomographic image. In addition, the imaging position determination unit 35 may determine a predetermined position in the body axis direction specified from the position P₃ of the waist, as the imaging position of the tomographic image. More specifically, a position separated from the position P₃ of the waist by a predetermined distance on the upper side (head side) may be determined as the imaging position.

In addition, in the above description, the case where the imaging system 1 is used for measuring the visceral fat of the subject has been described as an example, but the disclosed technology is not limited to this aspect. The disclosed technology can be applied to any examination, diagnosis, and the like using a tomographic image at a specific imaging position of the subject.

In the embodiments described above, for example, as a hardware structure of the processing units that execute various kinds of processing, such as the body thickness distribution derivation unit 32, the determination target position derivation unit 33, the reference position derivation unit 34, the imaging position determination unit 35, and the diagnosis information derivation unit 36, various processors illustrated below can be used. The above-described various processors include, for example, a programmable logic device (PLD) which is a processor having a changeable circuit configuration after manufacturing, such as an FPGA, and a dedicated electrical circuit which is a processor having a dedicated circuit configuration designed to execute specific processing, such as an application specific integrated circuit (ASIC), in addition to the GPU and the CPU which is a general-purpose processor that executes software (programs) to function as various processing units, as described above.

One processing unit may be configured by one of the various processors, or may be configured by a combination of the same or different types of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC), a form of using a processor that implements functions of the entire system including the plurality of processing units in one integrated circuit (IC) chip is possible. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

Moreover, in the embodiments described above, the aspect has been described in which the processing program 110 is stored (installed) in the non-volatile memory 103 in advance, but the disclosed technology is not limited to this. The processing program 110 may be provided in a form recorded in a recording medium such as a compact disc read-only memory (CD-ROM), a digital versatile disc read-only memory (DVD-ROM), and a universal serial bus (USB) memory. Alternatively, a form may be employed in which the above-described processing program 110 is downloaded from an external device via the network.

In regard to the first and second embodiments described above, the following additional remarks will be further disclosed.

### Supplementary Note 1

An information processing apparatus comprising:
at least one processor,
wherein the processor
   derives a determination target position of a subject in a body axis direction on the basis of body information indicating a physical feature of the subject in a predetermined range along the body axis direction of the subject,
   derives a reference position of the subject in the body axis direction on the basis of a subject image that is an image captured for the subject, and
   determines an imaging position of a tomographic image on the basis of a relative positional relationship between the determination target position and the reference position.

### Supplementary Note 2

The information processing apparatus according to Supplementary Note 1,
wherein the body information includes a body thickness distribution along the body axis direction of the subject, and
the processor derives a position at which a body thickness of the subject in the body axis direction specified from the body thickness distribution is a largest, as the determination target position.

### Supplementary Note 3

The information processing apparatus according to Supplementary Note 2,
wherein the processor
   acquires a distance image captured for the subject, and
   derives the body thickness distribution on the basis of the distance image.

### Supplementary Note 4

The information processing apparatus according to any one of Supplementary Notes 1 to 3,
wherein the processor
   acquires an optical image captured for the subject, as the body information, and
   derives a position of an umbilicus of the subject specified from the optical image, as the determination target position.

### Supplementary Note 5

The information processing apparatus according to any one of Supplementary Notes 1 to 4,
wherein the subject image is a scano image, and
the processor derives an uppermost portion of a pelvic region specified from the scano image in the body axis direction, as the reference position.

### Supplementary Note 6

The information processing apparatus according to any one of Supplementary Notes 1 to 4,
wherein the subject image is an optical image, and
the processor derives a position of a waist of the subject specified from the optical image, as the reference position.

### Supplementary Note 7

The information processing apparatus according to any one of Supplementary Notes 1 to 6,
wherein the processor
   determines a first position of the subject in the body axis direction as the imaging position in a case where the determination target position is on a lower side in the body axis direction with respect to the reference position, and
   determines a second position different from the first position as the imaging position in a case where the determination target position is on an upper side in the body axis direction with respect to the reference position.

### Supplementary Note 8

The information processing apparatus according to Supplementary Note 7,
wherein in a case where the determination target position is on the lower side in the body axis direction with respect to the reference position, the processor specifies a position of an anterior superior iliac spine and a position of a lower costal margin of the subject on the basis of the subject image, and determines a center between the position of the anterior superior iliac spine and the position of the lower costal margin in the body axis direction, as the imaging position.

### Supplementary Note 9

The information processing apparatus according to Supplementary Note 7,
wherein in a case where the determination target position is on the lower side in the body axis direction with respect to the reference position, the processor specifies a fourth lumbar vertebra of the subject on the basis of the subject image, and determines a center of the fourth lumbar vertebra in the body axis direction, as the imaging position.

### Supplementary Note 10

The information processing apparatus according to Supplementary Note 7,
wherein in a case where the determination target position is on the lower side in the body axis direction with respect to the reference position, the processor specifies a position of a waist of the subject on the basis of the subject image, and determines a predetermined position in the body axis direction specified from the position of the waist, as the imaging position.

### Supplementary Note 11

The information processing apparatus according to Supplementary Note 7,
wherein in a case where the determination target position is on the lower side in the body axis direction with respect to the reference position, the processor specifies a position of a shoulder and a position of a waist of the subject on the basis of the subject image, and determines a predetermined position in the body axis direction specified from both the position of the shoulder and the position of the waist, as the imaging position.

### Supplementary Note 12

The information processing apparatus according to Supplementary Note 7,
wherein in a case where the determination target position is on an upper side in the body axis direction with respect to the reference position, the processor determines the determination target position as the imaging position.

### Supplementary Note 13

An information processing method that is executed by at least one processor of an information processing apparatus, the information processing method comprising:
deriving a determination target position of a subject in a body axis direction on the basis of body information indicating a physical feature of the subject in a predetermined range along the body axis direction of the subject;
deriving a reference position of the subject in the body axis direction on the basis of a subject image that is an image captured for the subject; and
determining an imaging position of a tomographic image on the basis of a relative positional relationship between the determination target position and the reference position.

### Supplementary Note 14

A program for causing at least one processor of an information processing apparatus to execute processing comprising:
deriving a determination target position of a subject in a body axis direction on the basis of body information indicating a physical feature of the subject in a predetermined range along the body axis direction of the subject;
deriving a reference position of the subject in the body axis direction on the basis of a subject image that is an image captured for the subject; and
determining an imaging position of a tomographic image on the basis of a relative positional relationship between the determination target position and the reference position.

What is claimed is:

1. An information processing apparatus comprising:
at least one processor,
wherein the processor
derives a determination target position of a subject in a body axis direction on the basis of body information indicating a physical feature of the subject in a predetermined range along the body axis direction of the subject,
derives a reference position of the subject in the body axis direction on the basis of a subject image that is an image captured for the subject, and
determines an imaging position of a tomographic image on the basis of a relative positional relationship between the determination target position and the reference position.

2. The information processing apparatus according to claim 1,
wherein the body information includes a body thickness distribution along the body axis direction of the subject, and
the processor derives a position at which a body thickness of the subject in the body axis direction specified from the body thickness distribution is a largest, as the determination target position.

3. The information processing apparatus according to claim 2,
wherein the processor
acquires a distance image captured for the subject, and
derives the body thickness distribution on the basis of the distance image.

4. The information processing apparatus according to claim 1,
wherein the processor
acquires an optical image captured for the subject, as the body information, and
derives a position of an umbilicus of the subject specified from the optical image, as the determination target position.

5. The information processing apparatus according to claim 1,
wherein the subject image is a scano image, and
the processor derives an uppermost portion of a pelvic region specified from the scano image in the body axis direction, as the reference position.

6. The information processing apparatus according to claim 1,
wherein the subject image is an optical image, and
the processor derives a position of a waist of the subject specified from the optical image, as the reference position.

7. The information processing apparatus according to claim 1,
wherein the processor
determines a first position of the subject in the body axis direction as the imaging position in a case where the determination target position is on a lower side in the body axis direction with respect to the reference position, and
determines a second position different from the first position as the imaging position in a case where the determination target position is on an upper side in the body axis direction with respect to the reference position.

8. The information processing apparatus according to claim 7,
wherein in a case where the determination target position is on the lower side in the body axis direction with respect to the reference position, the processor specifies a position of an anterior superior iliac spine and a position of a lower costal margin of the subject on the basis of the subject image, and determines a center between the position of the anterior superior iliac spine and the position of the lower costal margin in the body axis direction, as the imaging position.

9. The information processing apparatus according to claim 7,
wherein in a case where the determination target position is on the lower side in the body axis direction with respect to the reference position, the processor specifies a fourth lumbar vertebra of the subject on the basis of the subject image, and determines a center of the fourth lumbar vertebra in the body axis direction, as the imaging position.

10. The information processing apparatus according to claim 7,
wherein in a case where the determination target position is on the lower side in the body axis direction with respect to the reference position, the processor specifies a position of a waist of the subject on the basis of the subject image, and determines a predetermined position in the body axis direction specified from the position of the waist, as the imaging position.

11. The information processing apparatus according to claim 7,
wherein in a case where the determination target position is on the lower side in the body axis direction with respect to the reference position, the processor specifies a position of a shoulder and a position of a waist of the subject on the basis of the subject image, and determines a predetermined position in the body axis direction specified from both the position of the shoulder and the position of the waist, as the imaging position.

12. The information processing apparatus according to claim 7,
wherein in a case where the determination target position is on an upper side in the body axis direction with respect to the reference position, the processor determines the determination target position as the imaging position.

13. An information processing method that is executed by at least one processor of an information processing apparatus, the information processing method comprising:
deriving a determination target position of a subject in a body axis direction on the basis of body information indicating a physical feature of the subject in a predetermined range along the body axis direction of the subject;
deriving a reference position of the subject in the body axis direction on the basis of a subject image that is an image captured for the subject; and
determining an imaging position of a tomographic image on the basis of a relative positional relationship between the determination target position and the reference position.

14. A non-transitory computer-readable storage medium storing a program for causing at least one processor of an information processing apparatus to execute processing comprising:
deriving a determination target position of a subject in a body axis direction on the basis of body information indicating a physical feature of the subject in a predetermined range along the body axis direction of the subject;
deriving a reference position of the subject in the body axis direction on the basis of a subject image that is an image captured for the subject; and
determining an imaging position of a tomographic image on the basis of a relative positional relationship between the determination target position and the reference position.
